(19) United States
(12) Patent Application Publication
(10) Pub. No.: US 2007/0154426 A1
(43) Pub. Date: Jul. 5, 2007

(54) Powdered water cosmetic compositions and related methods

(76) Inventors: Angela M. Hansen, Edison, NJ (US); Julio Gans Russ, Westfield, NJ (US); Kalliope Sappho Lordi, Clark, NJ (US)

Correspondence Address: JULIE BLACKBURN;REVLON CONSUMER PRODUCTS CORPORATION, 237 PARK AVENUE, NEW YORK, NY, 10017, US

(21) Appl. No.: 11/326,150

(22) Filed: Jan. 5, 2006

(51) Int. Cl. A61K 8/19

(52) U.S. Cl. 424/63; 424/64

(57) ABSTRACT

A powdered water color cosmetic composition comprising a water phase comprised of water droplets, and a particulate phase comprised of non-wetting particulates operable to surround and sequester the individual water droplets from each other in the composition, and a stabilizing effective amount of at least one water insoluble super absorbent polymer.

### TECHNICAL FIELD

The invention is in the field of aqueous based color cosmetic compositions powdered water form.

### BACKGROUND OF INVENTION

The term “powdered water” or “dry water” refers to compositions that exist in the dry powder form yet contain appreciable amounts of water. Powdered water compositions are typically made by combining water or water soluble solvents with certain hydrophobic non-wetting particulate materials such as silica. When the water is mixed with the particulates, the particulates surround each water droplet and sequester it from the other particulate surrounded water droplets. The resulting composition is in a powder form. However, when compressed, the water will exude from the particulate coating and liquefy on contact with the application surface. Powdered water was discovered in the 1960's and has been used in cosmetics.

U.S. Pat. No. 3,393,155 teaches compositions that are predominantly aqueous based but exist in a dry powder form and methods for their preparation.

U.S. Patent Publication No. 2004/0028719A1, teaches another method for preparing dry water, which involves agitating water and hydrophobic particulates together to form a powdered water composition. The patentee teaches that other ingredients may be present such as water soluble polymers in the aqueous phase.

U.S. Pat. No. 6,290,941 teaches liquid to powder cosmetic compositions in such a form. Patentee teaches that such powder to liquid compositions contain hydrophobically coated silica particles into which are incorporated water and a water soluble polymer. However, one problem with such compositions is that the water tends to seep out of the particles on storage. Water leakage causes the composition to become paste like with time. These problems are believed to be due to the fact that the water absorbent polymer present in the compositions is water soluble.

While powdered water compositions are very desirable for use in cosmetics, because of their instability and the difficulties associated with formulation they do not serve as the basis for many commercial products.

Accordingly, it has been discovered that when a water insoluble super absorbent polymer is included in the particulate phase of dry water compositions, the compositions are substantially more stable and comfortable. The super absorbent polymer resides in the particulate phase that surrounds the water droplets and acts to absorb any water that may leach out of the particulate-surrounded water droplets. This, in turn, stabilizes the composition.

It is an object of the invention to provide powdered water color cosmetic compositions that are stable and aesthetically pleasing.

It is a further object of the invention to provide color cosmetic compositions in the powdered water form that are suitable for use as lipsticks, eyeshadows, foundations, or powders.

It is a further object of the invention to provide color cosmetic compositions in the powdered water form that may be used to deliver active ingredient to the skin or lips.

### SUMMARY OF THE INVENTION

The invention is directed to a powdered water color cosmetic composition comprising a water phase comprised of water droplets, and a particulate phase comprised of (i) non-wetting particulates operable to surround and sequester the individual water droplets from each other in the composition, and (ii) a stabilizing effective amount of at least one water insoluble super absorbent polymer.

### DETAILED DESCRIPTION

I. Definitions

A. Non-Wetting, with respect to the particulates, means that they are not wet by, or solvated with, water.

B. Percentages. All percentages mentioned herein are percentages by weight unless otherwise indicated.

C. Powdered Water or Dry Water. Refers to compositions that contain water but where the coalescence of the individual water droplets to form liquid water is prevented because the individual water droplets are surrounded with non-wetting hydrophobic particulates in an amount sufficient to sequester the water droplets from each other while the composition is in the resting state.

D. Wettability means the ability of a solid surface (in this case the non-wetting particulate) to be wet by the water droplets comprising the water phase of the composition. The degree of wetting is generally determined by measurement of the contact angle between the solid and liquid phases when both are at thermal equilibrium.

II. The Composition

The powdered water compositions of the invention are color cosmetics comprising the following ingredients.

A. Water

The powdered water compositions of the invention generally contain from about 1-65%, preferably from about 2-60%, more preferably from about 5-55% by weight water. In the compositions of the invention, the individual water droplets present may have particle sizes ranging from about 5 to 200 microns, preferably from about 8 to 100, more preferably from about 10 to 80 microns.

B. Non-Wetting Particulates

The powdered water compositions of the invention comprise from about 0.1-85%, preferably from about 0.5-80%, more preferably from about 1-75% by weight of the total composition of non-wetting particulates, preferably silica. Such particulates may have particle sizes ranging from about 0.005 to 75 microns.

Suitable non-wetting particulates are silicas or fumed silicas that tend to be hydrophobic in nature. In general, such silicas are made by vapor phase hydrolysis of silicon tetrachloride vapor in a hydrogen oxygen flame. The resulting combustion process creates silicon dioxide molecules which condense to form particles, which in turn collide, attach, and sinter together to form a three dimensional network. In many cases the resulting silica has considerable hydroxyl functionality, meaning that it will be hydrophilic in nature. In order to make the silica hydrophobic for certain desired applications, the silica may be substituted with C₁₋₆ alkyl or alkoxy groups, in particular methyl, ethyl, or propyl groups. Suitable hydrophobic silicas include those that are surface treated or bonded with silicones such as dimethicone or methicone, or where the silica is prepared in such a manner that it has an alkyl or alkoxy functionality sufficient to provide the desired hydrophobic character.

Examples of suitable hydrophobic silicas are sold by DeGussa under the Aerosil® brand, more specifically Aerosil® R, which indicates hydrophobic character. Included within the Aerosil® R silicas suitable for use in the compositions of the invention are Aerosil® R 202, 972, 974, 805, 812S, and so on. Most preferred is Aerosil® R202, a hydrophobic fumed silica after treated with polydimethylsiloxane. Suitable hydrophobic silicas are also sold by Cabot Corporation under the Cab-O-Sil® brand such as Cab-O-Sil® TS-530 which is silica treated with hexamethyldisilazane, Cab-O-Sil® TS-720 which is silica treated with dimethicone, and so on.

The non-wetting particulates are capable of surrounding a water droplet to form a coating thereon which is sufficiently strong to prevent the water droplets present in the composition from coalescing to form liquid water.

C. Water Insoluble Super Absorbent Polymer

The compositions of the invention also contain at least one water insoluble super absorbent polymer. Preferably this polymer is a crosslinked polyacrylate, more particularly crosslinked sodium polyacryate. While sodium polyacrylates are well known in the art, they tend to be uncrosslinked, which makes them highly water soluble. Most suitable are sodium polyacrylates sold under the brand name Aqua-Keep® by Kobo Products, Inc. including Aqua-Keep® J-440, SAGON Type II, J-550, or 10SH-NF. Particularly preferred is sodium polyacrylate sold by Kobo Products under the brand name Aqua-Keep® 10SH-NF, which has an absorption capacity of about 60 grams per gram of material, an absorption rate of about 8 sec., a bulk density of about 0.87 g/ml, with a particle size of under 100 microns.

The water insoluble super absorbent polymer used in the compositions of the invention is dispersed in the particulate phase. When sufficient hydrophobic silica particles surround the water droplet such that it is prevented from coalescing, the water insoluble super absorbent polymer that remains in the particulate phase. Should any water leach out of the water droplets surrounded by silica, it will be absorbed by the polymer. This improves the stability of the composition.

D. Pigments and Powders

The composition of the invention preferably contains pigments, powders or both. Suggested ranges are from about 0.1 to 95%, preferably from about 0.5-85%, more preferably from about 1-70% by weight of the total composition. The pigments or powders or both may be coated with one or more ingredients that confer hydrophobicity.

1. Pigments

Suitable pigments include organic or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

2. Powders

Suitable powders include colored or non-colored (for example white) non-pigmentatious particles. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, polymethylsilsesquioxane, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof.

The above mentioned pigments and powders may be surface treated with lecithin, amino acids, mineral oil, silicone, lauroyl lysine, fluoro compounds, or various other agents either alone or in combination, which coat the powder surface and render the particles more lipophilic in nature. Preferred is where one or more of the pigments and/or powders present are coated with ingredients that confer such hydrophobicity. Particularly preferred are fluoro compounds, more specifically fluorinated paraffinic hydrocarbons or alcohols, such as C9-15 fluoroalcohols, and the like.

In the most preferred embodiment of the invention, more than 75%, preferably more than 80% of the pigments, powders, or both are hydrophobic, or coated with sufficient hydrophobic material to confer hydrophobicity.

E. Other Ingredients

If desired, the compositions of the invention may contain other ingredients such as humectants, preservatives, botanical ingredients and the like.

1. Humectants, Stablizers

If present, suggested ranges of humectant are from about 0.01-30%, preferably 0.5-25%, more preferably 1-20% by weight of the total composition. In general, such humectants are water soluble and will be found in the water phase droplets in the composition of the invention. Suitable humectants include materials such as glycols, sugars, and the like. Suitable glycols include polyethylene and polypropylene glycols such as PEG 4-240, which are polyethylene glycols having from 4 to 240 repeating ethylene oxide units; as well as C₁₋₆ alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

Stabilizers may also be added, which are ingredients that stabilize the composition, particularly the water phase. Examples are water soluble salts and urea or urea derivatives. Stabilizers, if present, may be found in the same ranges as humectants.

2. Botanical Extracts

It may be desirable to include one or more botanical extracts in the compositions. If so, suggested ranges are from about 0.0001 to 10%, preferably about 0.0005 to 8%, more preferably about 0.001 to 5% by weight of the total composition. Suitable botanical extracts are preferably water soluble and will be solubilized in the water droplets. Such extracts include those from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, and mixtures thereof.

3. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

The powdered water compositions are prepared by combining the silica and the water phase ingredients and mixing well in a blender at high speed. The remaining ingredients are added to the pre-mix and blended at high speed in the blender.

The resulting compositions are in the powdered form. When applied to skin, the non-wetting particulates that surround the water droplets and prevent them from coalescing into liquid water when the composition is in the resting state, will disperse so that the water present is applied to the skin along with the pigments and powders present. The compositions provide a very light, non-greasy, color to the skin. The composition may be in the form of a foundation, powder, eye shadow, lipstick, blush, or any other type of color cosmetic product.

The invention will be further described in connection with the following examples which are set forth for the purposes of illustration only.

### EXAMPLE 1

A powdered water composition suitable as a face powder or foundation makeup was prepared as follows:

| | | % by |
|---|---|---|
| | Ingredient | weight |
| 1 | Talc, C9-15 fluoroalcohol phosphates | 34.1 |
| 2 | Methyl paraben | 0.20 |
| 3 | Boron nitride, fluoroalcohol phosphate | 1.00 |
| 4 | Propyl paraben | 0.10 |
| 5 | Red iron oxides, C9-15 fluoroalcohol phosphates | 0.15 |
| 6 | Yellow iron oxides, C9-15 fluoroalcohol phosphates | 0.25 |
| 7 | Black iron oxides, C9-15 fluoroalcohol phosphates | 1.00 |
| 8 | Titanium dioxide, C9-15 fluoroalcohol phosphate | 5.00 |
| 9 | Sodium polyacrylate* | 0.50 |
| 10 | Lauroyl lysine | 1.00 |
| 11 | Polymethylsilsesquioxane (GE Silicones, Tospearl) | 1.00 |
| 12 | Water | 46.00 |
| 13 | Sodium chloride | 2.00 |
| 14 | Diazolidinyl urea | 0.20 |
| 15 | Butylene glycol | 2.50 |
| 16 | Silica, dimethicone (Aerosil ® R202) | 5.00 |

*cross-linked, water insoluble sodium polyacrylate, Aqua-Keep 10SH-NF, Kobo Products Inc.

The water phase ingredients (12-15) were combined and mixed with the Aerosil R202 (16) in a kitchen blender on high speed for 4 minutes. Separately, ingredients 1-11 were combined and mixed in a blender on high speed for 1.5 minutes. After stopping the blender and scraping the sides, the mixture was mixed on high speed for another 1.5 minutes. The pre-mix of ingredients 12-16 was added to the mixture of ingredients 1-11 and mixed on low for 30 seconds. The resulting composition was a powder.

### EXAMPLE 2

Other color cosmetic compositions were made as follows:

| | % by weight | | | |
|---|---|---|---|---|
| | Bronze | | Eye | Lip |
| Ingredient | Powder | Foundation | shadow | Powder |
| Talc, C9-15 fluoroalcohol phosphate | QS | QS | QS | QS |
| Methyl paraben | 0.20 | 0.20 | 0.20 | 0.20 |
| Mica, iron oxide, titanium dioxide, C9-15 fluoroalcohol | — | — | 1.00 | 1.00 |
| phosphate | | | | |
| Propyl paraben | 0.10 | 0.10 | 0.10 | 0.10 |
| Red iron oxides, C9-15 fluoroalcohol phosphates | 0.15 | | 0.15 | 0.15 |
| Yellow iron oxides, C9-15 fluoroalcohol phosphates | 0.25 | | 0.25 | 0.25 |
| Black iron oxides, C9-15 fluoroalcohol phosphates | 1.00 | | 1.00 | 1.00 |
| Sodium polyacrylate* | 0.50 | 0.50 | 0.50 | 0.50 |
| Lauroyl lysine | 1.00 | 1.00 | 1.00 | 1.00 |
| Polymethylsilsesquioxane (GE Silicones, Tospearl) | 1.00 | 1.00 | 1.00 | 1.00 |
| Mica, iron oxides, titanium dioxide, C9-15 fluoroalcohol | 15.00 | 3.00 | 15.00 | 15.00 |
| phosphate | | | | |
| Ultramarine blue, C9-15 fluoroalcohol phosphate | — | — | 0.25 | — |
| Titanium dioxide, C9-15 fluoroalcohol phosphate | — | 3.00 | — | 15.00 |
| Yellow #5 Lake | — | — | 0.25 | 0.10 |
| Yellow #6 Lake | — | — | — | 0.10 |
| Red #33 | — | — | — | 0.10 |
| Red #40 | — | — | — | 0.10 |
| Blue Lake | — | — | — | 0.10 |
| Water | 46.00 | 46.00 | 46.00 | 46.00 |
| Sodium chloride | 2.00 | 2.00 | 2.00 | 2.00 |
| Diazolidinyl urea | 0.20 | 0.20 | 0.20 | 0.20 |
| Butylene glycol | 2.50 | 2.50 | 2.50 | 2.50 |
| Silica, dimethicone (Aerosil ® R202) | 5.00 | 5.00 | 5.00 | 5.00 |

*cross-linked, water insoluble sodium polyacrylate, Aqua-Keep 10SH-NF, Kobo Products Inc.,

The compositions were made as set forth in Example 1.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A powdered water color cosmetic composition comprising a water phase comprised of water droplets, and a particulate phase comprised of (i) non-wetting particulates operable to surround and sequester the individual water droplets from each other in the composition, and (ii) a stabilizing effective amount of at least one water insoluble super absorbent polymer.

2. The composition of claim 1 wherein the non-wetting particulates are hydrophobic silica.

3. The composition of claim 3 wherein the hydrophobic silica is silica substituted with C1-6 alkyl or alkoxy groups.

4. The composition of claim 2 wherein the hydrophobic silica is silica substituted with methicone.

5. The composition of claim 2 wherein the hydrophobic silica is silica coated with dimethicone.

6. The composition of claim 1 wherein the water insoluble super absorbent polymer is crosslinked sodium polyacrylate.

7. The composition of claim 1 further comprising from about 0.1 to 95% pigments or powders.

8. The composition of claim 7 wherein the pigments or powders are coated with a hydrophobic ingredient.

9. The composition of claim 8 wherein the hydrophobic ingredient is silicone, mineral oil, silk, or a fluoroalcohol.

10. The composition of claim 9 wherein the hydrophobic material is C9-15 fluoroalcohol.

11. The composition of claim 1 comprising, by weight of the total composition, from about 0.1-85% non-wetting particulates, from about 1-65% water, and from about 1-95% of pigments or powders.

12. The composition of claim 11 wherein the non-wetting particulates are silica treated with dimethicone.

13. The composition of claim 11 additionally comprising from about 0.1-20% humectant in the water phase.

14. The composition of claim 11 comprising from about 0.1-50% pigment.

15. The composition of claim 14 wherein the pigments are one or more iron oxides.

16. The composition of claim 14 wherein the powders include mica, titanium dioxide, polymethylsilsesquioxane, boron nitride, or mixtures thereof.

17. The composition of claim 1 which is a foundation makeup.

18. The composition of claim 1 which is an eye shadow.

19. The composition of claim 1 which is a powder.

20. The composition of claim 1 which is a lipstick.
